(19) United States
(12) Patent Application Publication
(10) Pub. No.: US 2025/0161070 A1
(43) Pub. Date: May 22, 2025

(54) SACROILIAC JOINT STABILIZATION, INCLUDING IMPLANTS, SYSTEMS AND METHODS OF DELIVERING IMPLANTS

(71) Applicant: SI-Bone Inc., Santa Clara, CA (US)

(72) Inventors: Paul M. SAND, Redwood City, CA (US); Bret SCHNEIDER, San Jose, CA (US); Derek P. LINDSEY, San Jose, CA (US); Francois FOLLINI, Austin, TX (US); Nikolas F. KERR, Milpitas, CA (US); Roxanne SIMON, San Jose, CA (US); Scott A. YERBY, Montara, CA (US)

(21) Appl. No.: 18/951,396

(22) Filed: Nov. 18, 2024

Related U.S. Application Data
(60) Provisional application No. 63/600,475, filed on Nov. 17, 2023.

(51) Int. Cl. A61F 2/46; A61F 2/30

(52) U.S. Cl. CPC A61F 2/4603; A61F 2002/30995

(57) ABSTRACT

Methods of and instruments for positioning a sacroiliac joint implant in a dorsal trajectory down the joint. The methods may include positioning an integrated stylet and cannula adjacent the joint, removing the stylet from a cannula lumen, and advancing the implant through the cannula lumen with tamp and into the joint.

### CROSS REFERENCE TO RELATED APPLICATIONS

This application claims the benefit of U.S. Provisional App. No. 63/600,475, filed Nov. 17, 2023, which is fully incorporated by reference herein for all purposes.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

It would be beneficial for SI joint implantation procedures to require fewer instruments, tools and steps than existing procedures, particularly for dorsal, down-the-joint, deliveries, and particularly for bone dowel implants.

### SUMMARY OF THE DISCLOSURE

One aspect of the disclosure is a method of positioning a sacroiliac (SI) joint implant, comprising: in a dorsal trajectory, positioning a stylet removably coupled with a cannula adjacent to an SI joint, the stylet disposed through a cannula lumen with sharp distal tip of the stylet 20 mm-40 mm distally from a cannula distal end; advancing, in the dorsal trajectory, the sharp distal end of the stylet down and into an SI joint, while a portion of the stylet is within the cannula lumen, to disrupt the SI joint and prepares the SI joint to receive an SI joint implant; removing the stylet from the SI joint and from the cannula lumen, leaving the cannula distal end adjacent to the SI joint; positioning an SI joint implant into the cannula lumen; advancing the SI joint implant with a plunger distally through the cannula lumen and into the SI joint; removing the plunger from the cannula; and removing the cannula from the subject while leaving the implant in the SI joint.

One aspect of the disclosure is a method of positioning a sacroiliac (SI) joint implant, comprising: advancing, in a dorsal approach, a cannula to a level of an SI joint; advancing a sharp distal end of a stylet down the SI joint while a portion of the stylet is within a cannula lumen, wherein a distal tip of the stylet is positioned 20 mm-40 mm distally from a cannula distal end, and wherein advancing the sharp distal end of the stylet down the SI joint disrupts the SI joint and prepares the SI joint to receive an SI joint implant; removing the stylet from the cannula lumen and leaving the cannula adjacent to the SI joint; positioning an SI joint implant into the cannula lumen; advancing the SI joint implant distally through the cannula lumen and down and into the SI joint; and removing the cannula from the subject while leaving the implant in the patient.

One aspect of the disclosure is a system for implanting a sacroiliac (SI) joint implant down the SI joint in a dorsal approach, comprising: a cannula having a lumen and a sharpened stylet removably couplable to the cannula, the cannula and stylet are each sized and configured such that, when coupled, a portion of the stylet is within the cannula lumen and a distal tip is 20 mm to 40 mm distal to a distal end of the cannula.

One aspect of the disclosure is a cannula stabilization and stylet removal tool, comprising: an elongate body adapted to be held by a hand of an operator; a clamp end comprising a first jaw movable relative to a second jaw about a pivot, the first jaw including a first locking element; a locking collar axially movable relative to the elongate body, the locking collar including a second locking element sized and configured to interface with the first locking element to maintain the collar in a locked position relative to the elongate body and prevent clamp ends of the first and second jaws from moving away from one another; and a lever end opposite the clamp end, the lever end comprising first and second hooks each having a curved configuration including a recessed surface, the curved first and second hooks extending from the elongate body, and each of the first and second hooks sized and configured to accommodate and interface with a proximal handle portion of a stylet to facilitate application of a removal force to the proximal handle portion of the stylet.

### BRIEF DESCRIPTION OF THE DRAWINGS

FIG. 1 illustrates a prior art set of instruments, including more components and complexity that approaches and instrument sets herein.

FIG. 2 illustrates an exemplary set of instruments for delivering an SI joint implant.

FIG. 3 illustrates an exemplary set of instruments for delivering an SI joint implant.

FIG. 4 illustrates an exemplary set of instruments for delivering an SI joint implant.

FIGS. 5A and 5B illustrate an exemplary integrated cannula and stylet.

FIGS. 6A and 6B illustrate an exemplary SI joint implant.

FIG. 7 illustrates an exemplary distal region of an exemplary stylet.

FIG. 8 illustrates an exemplary patient position for procedures herein.

FIGS. 9, 10, 11, 12 and 13 illustrate exemplary planning for the SI joint implant implantation.

FIGS. 14A and 14B illustrate an exemplary trajectory for the stylet/cannula.

FIG. 15A illustrates a preferred trajectory and position, compared with FIG. 15B.

FIG. 16A illustrates an exemplary optional stabilization tool with a clamp end.

FIG. 16B illustrates the exemplary optional stabilization tool from FIG. 16A clamp around a cannula.

FIGS. 17A and 17B illustrate views related to confirming a trajectory of the stylet.

FIG. 18 represents a position and trajectory of an integrated cannula/stylet with a distal region of the stylet/cannula within the inferior limb of the SI joint.

FIGS. 19A, 19B and 19C illustrate a stylet loosening/removal process.

FIGS. 20A and 20B illustrate using a lever end of a tool to remove a stylet.

FIG. 21 illustrates positioning an implant into a cannula lumen followed by a tamp.

FIG. 22 illustrates advancing a tamp to distally advance the implant.

FIG. 23 illustrates an exemplary preferred position of an SI joint implant.

FIGS. 24A and 24B illustrate an exemplary trajectory for a second implant.

FIG. 25 illustrates stylet advanced parallel to a first implant.

FIGS. 26A, 26B and 26C illustrate first and second implants positioned in an SI joint.

### DETAILED DESCRIPTION

The patent or application file contains at least one drawing executed in color. Copies of this patent or patent application publication with color drawing(s) will be provided by the Office upon request and payment of the necessary fee.

There may be a need to stabilize and/or fuse one or more of a subject's sacroiliac (SI) joints. Methods herein include methods of delivering stabilizing and/or fusing implants to the SI joint, wherein when implanted stabilize the implant over time. The terms stabilizing and fusing (and derivatives thereof) may both be referred to herein as “stabilizing” or “stabilization” (or derivatives thereof). Implant procedures herein describe dorsal, or posterior, approaches for accessing, preparing the joint, and delivering the implant, all of which are considered herein as dorsal approaches. The dorsal approaches described herein comprise delivering implants “down the joint.” Implants herein may also be described as being implanted in a dorsal or posterior manner. The entire disclosure of US20230000639A1, including dorsal/posterior approaches and trajectories is incorporated by reference herein and may be used at least in part in the dorsal approaches and trajectories for delivering the SI joint implants herein.

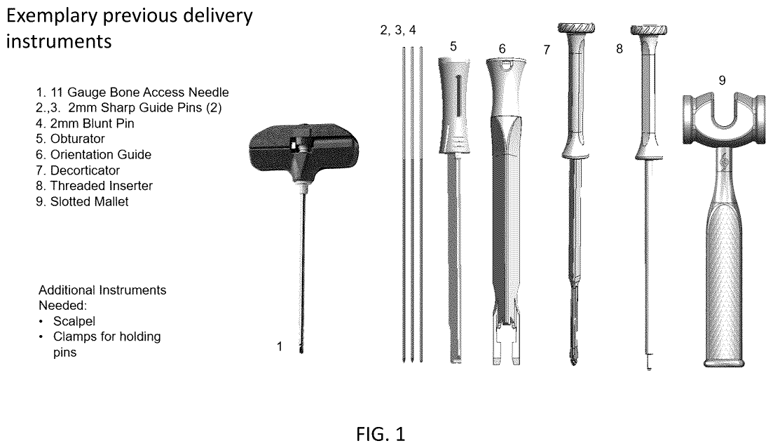

One of the advantages of the implantation methods, system, and devices described herein is that they require fewer steps and fewer delivery components compared to other SI joint stabilization procedures that utilize a dorsal, “down the joint” approach. Reducing the number of components and steps shortens the procedure time, reduces the likelihood of component malfunction, reduces the risk of patient injury, reduces cost, and requires less training. For example, methods herein eliminate the need to utilize an obturator, and orientation or placement guide, and/or a decorticator, for example, examples of which are illustrated in FIG. 1.

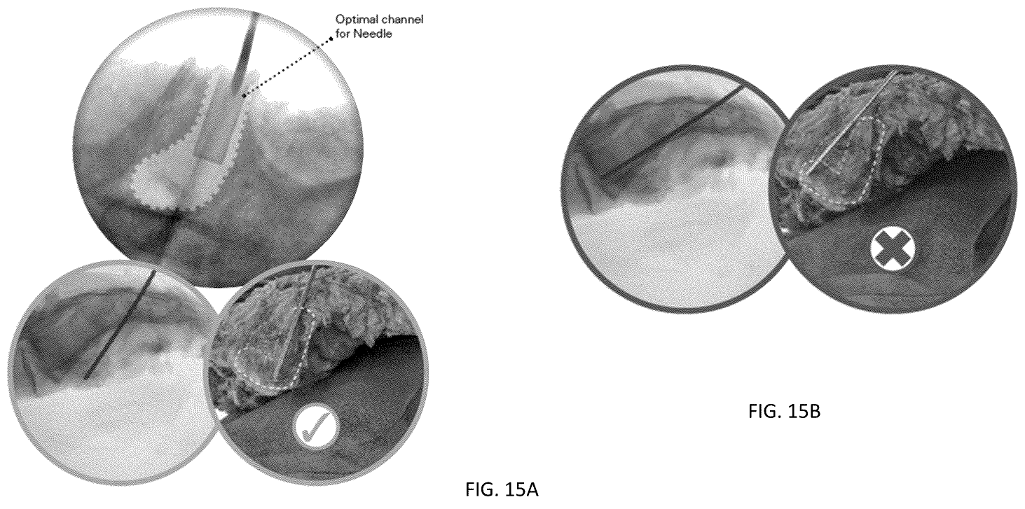

An additional non-limiting advantage of the innovative dorsal approaches and systems described herein is that the trajectory is slightly different than previous dorsal approaches. For example, trajectories herein are inferior, or lower than, previous approaches, as example of which is shown in the preferred trajectory of FIG. 15A compared with the non-preferred trajectory shown in FIG. 15B. FIG. 15B illustrates a less than preferred trajectory, which is more superior compared with the trajectory illustrated in FIG. 15A, and which passes through, or includes a higher probability of passing through, the ligamentous portion of the SI joint. Any of the methods herein that advance a sharpened stylet down the joint may therefore include advancing the stylet in the trajectory shown in FIG. 15A, which is through the articular portion of the joint rather than the ligamentous portion of the joint. The trajectory shown in FIG. 15A is also referred to as being aligned with the ventral aspect of the articular joint. The trajectory shown in FIG. 15A (which is the same trajectory for pain injections, so which may be more familiar to physicians) may provide for better bone seating and anchoring when the implant is implanted.

While the disclosure herein focuses on dorsal approach, methods and systems described herein may find application in lateral implantation methods, wherein the implant is implanted laterally across the SI joint, across an ilium, across the SI joint, and into the sacrum.

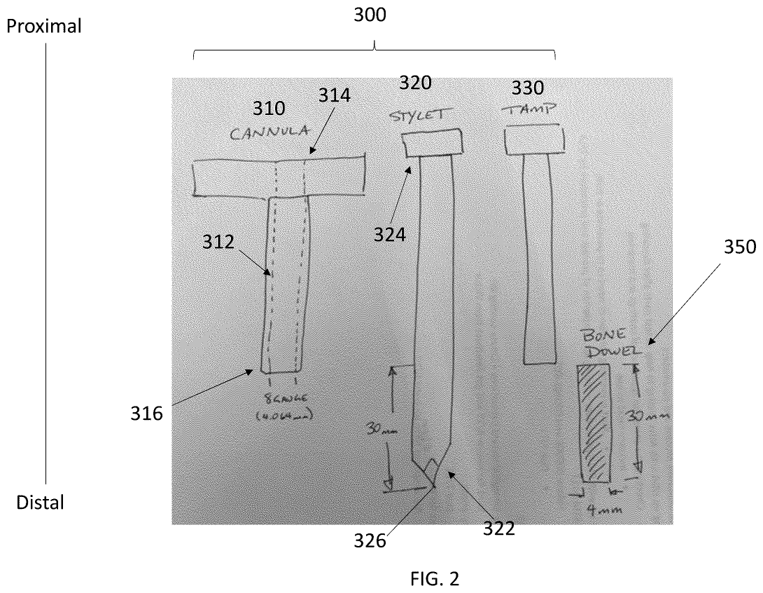

FIG. 2 illustrates exemplary instruments 300 for one or more of accessing an SI joint, preparing an SI joint for an implant, and delivering an implant down the SI joint. Relative proximal and distal directions are labeled. Instruments 300 include cannula 310 with internal lumen 312 therethrough from a proximal end to distal end 316; stylet 320 with a sharpened distal end sized and configured to be disposed within and removable from cannula lumen 312, with a sharpened distal end 322 for entry into and down the SI joint to prepare the SI joint for the implant; and an implant plunger or tamp 330 sized and configured to (after the stylet is removed from cannula lumen 312) advancing (optionally pushing distally) the implant 350 distally through cannula lumen 312 and into the SI joint.

FIG. 2 illustrates an exemplary SI joint stabilization implant or bone dowel 350, with exemplary and non-limiting dimensions provided, which is configured to be advanced distally through cannula lumen 312 and into an SI joint.

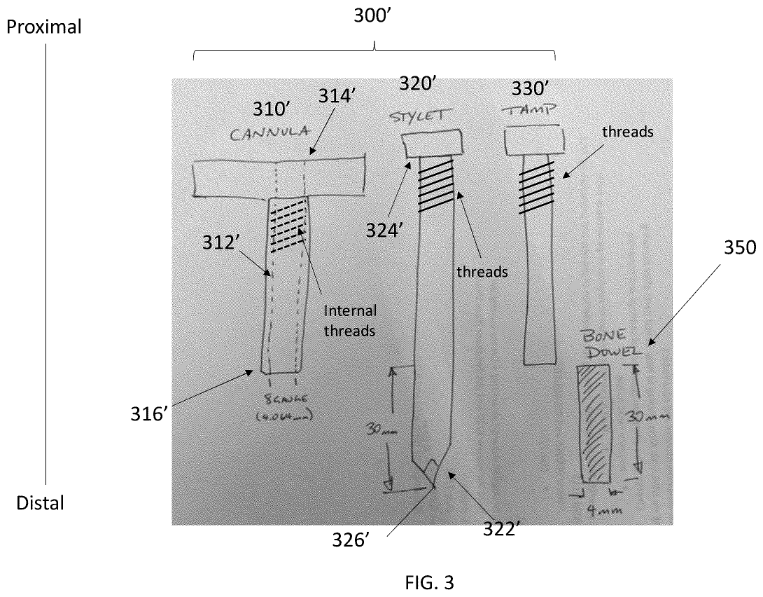

FIG. 3 illustrates alternative instruments 300′ with components labeled similarly as in the system of FIG. 2. The stylets herein can be removably coupled to the cannulas herein in a variety of ways. For example, the stylets herein may optionally have outer threads shaped to mate with inner threads on the cannula lumen, such as shown in FIG. 3. This arrangement can allow the stylet to be removed by rotating the stylet relative to the cannula, compared with an axially directed proximal pulling force out of the SI joint, which is described with systems below. The stylets herein may also include at least one proximal region surface (e.g., surface 324′), and the cannula may include at least one proximal end surface (e.g., surface 314′), wherein the at least one proximal end surfaces on the stylet and the cannula are sized and configured to engage to prevent further distal stylet movement and maintain the relative distal positions of the cannula and the stylet when engaged, which may cause the distal tip (e.g., 326′) of the stylet to be 20 mm-40 mm distally beyond a cannula distal end.

Similarly, the plungers described herein may have outer threads sized and configured to interface with a cannula lumen inner threads, such as shown in FIG. 3, which may facilitate advancing the implant upon rotation of the plunger relative to the cannula (and to retract the plunger).

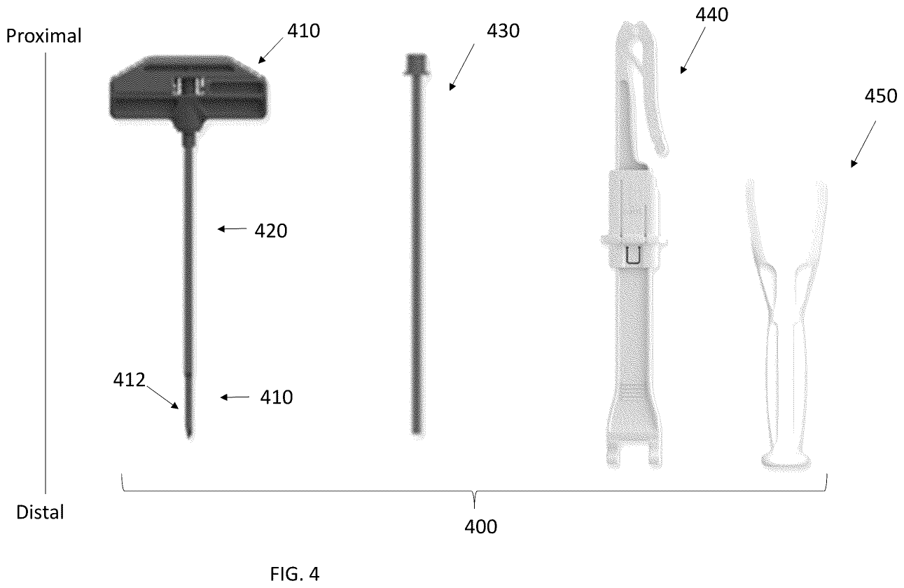

FIG. 4 illustrates an alternative system of instruments 400 that includes stylet 410, cannula 420, tamp or plunger 430, optional stabilization and lever tool 440, and mallet 450. An implant 460 that may be implanted with system 400 is shown in the end view of FIG. 6A and side view of FIG. 6B, although other implants may be implanted with system 400. Stylet 410 is shown within cannula 420, with the distal region 412 of stylet shown extending distally beyond the distal end of cannula. Use of instruments 400 is described in more detail herein. When the stylet is positioned within the cannula (e.g., as in FIG. 4), the stylet and cannula may be referred to herein as, for example, “integrated,” “combined,” “assembled” and/or may together be referred to as a “needle.”

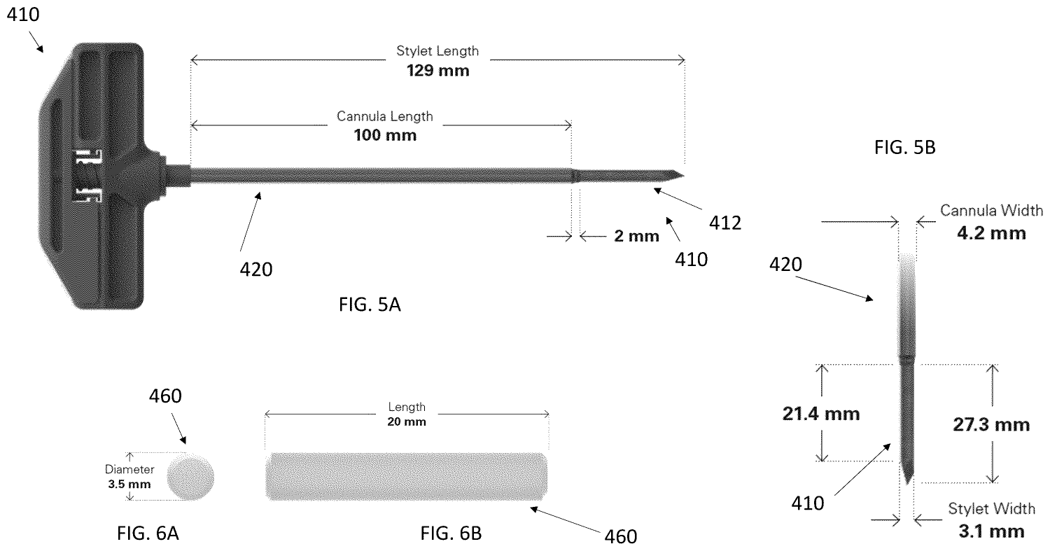

Additional exemplary and non-limiting features of stylet and cannula are shown in FIGS. 5A and 5B, where FIG. 5B illustrates a distal portion of the combined cannula and stylet.

The stylets described herein have a length that is greater than a length of the corresponding cannula in which the stylet is disposed. The stylets described herein have a sharp or sharpened distal end (e.g., distal end 322) that includes a distal tip (e.g., distal tip 326), wherein the distal tip is the furthest distal location on the stylet. The lengths are measure proximal to distal, with exemplary lengths labeled in FIG. 5A for reference. In general, the stylets herein prepare the joint for the implant (e.g., bone dowel). The stylets herein may have a smooth outer surface. Alternatively, the stylets herein may include one or more abrasive and/or rasping elements on an outer surface thereof that are adapted to further disrupt the SI joint (further disrupt considered relative to the sharped distal end).

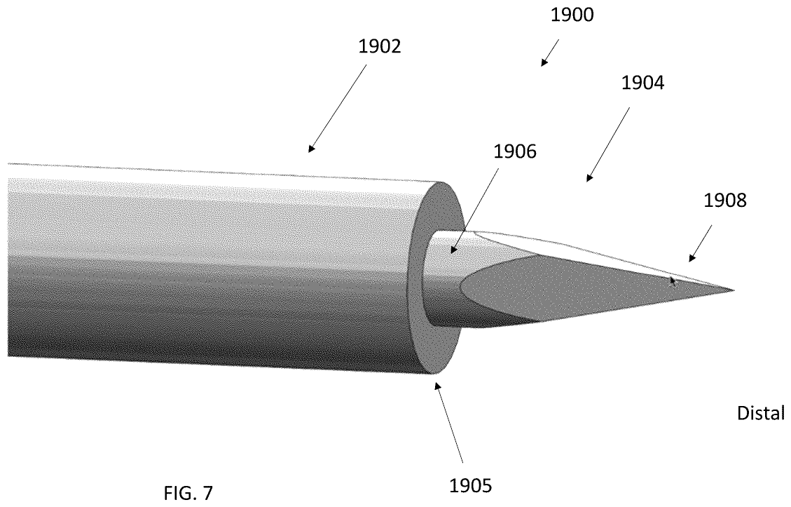

FIG. 7 illustrates an exemplary alternative stylet 1900, which may be used with and/or integrated into any of the instruments herein. As shown, the distal end of the stylet 1900 includes a region with step 1905, so the square (or other step which may have a non-90 degree angle) shoulder of step 1905 is adapted to, and will when advanced, scrape cartilage down the joint, so that the implant can be implanted against bleeding bone. Tip 1906 may optionally be 2 mm in diameter with a 15 degree 4-sided trocar 1908.

Plungers or tamps described herein (e.g., plungers 330, 430) are generally sized and configured to fit within the cannula lumen and advance the implant therethrough, optionally upon a proximal force applied to the plunger (e.g., with a mallet) and indirectly to the implant through the plunger.

The SI joint implants described herein may have a variety of sizes and configurations. The implants may have a cylindrical configuration, optionally with short bevels at one or both of the implant ends (e.g., as with implant 460). Cylindrically configured implants may be easier to manufacture than other configurations. The implants may have oval, or square (or other rectilinear) cross sectional shapes (in a plane transverse to the implant long axis).

The implants herein may be sized to be implanted solely in the SI joint, or they may be sized and configured to be implanted such that they extend into the sacral and/or iliac bones adjacent the SI joint to some extent.

The implants herein may have smooth outer surfaces. The implants herein may have porous outer surfaces, which are optionally coatings or a result of a manufacturing process.

The implants herein may have the same outer diameter (“OD”) as the stylet. The implants herein may have an OD greater than or less than the OD of the stylet. The implants may have ODs just slightly less than diameter of the cannula lumen (which may be an inner diameter even if there are internal surface threads on the cannula lumen).

The implants herein may be cylinders of bone with a central lumen extending therethrough.

The implant herein may be allografts, may be ceramic, may be alloys, may comprise hydroxyapatite, may comprise calcium phosphate, and/or may comprise one or more polymeric materials.

In some examples the implants herein may be 15 mm to 35 mm in length (from proximal end to distal end), wherein implant 460 is an example of such a length range. In some examples the implants herein may have an outer diameter from 3.0 mm to 4.5 mm, optionally from 3.2 mm to 4.1 mm, wherein implant 460 is an example of such a diameter range.

A general method of implanting an SI joint stabilizing implant (such as implant 350 or 460) includes positioning a cannula (e.g., 310) removably coupled with a stylet (e.g., 320, or as shown in FIGS. 4 and 5) adjacent to an SI joint, the stylet disposed through a cannula lumen (e.g., 312) with a sharp distal end (e.g., 322) of the stylet, including a distal tip 326, extending 20 mm-40 mm (optionally 30 mm) distally from a cannula distal end (e.g., 316); advancing, in a dorsal approach, the sharp distal end of the stylet down and into the SI joint while the stylet is within the cannula lumen, wherein advancing the sharp distal end of the stylet into and down the SI joint disrupts the SI joint, preparing the SI joint to receive an SI joint implant; withdrawing the stylet from the cannula lumen while maintaining the cannula in position adjacent to the SI joint; positioning an SI joint implant into the cannula lumen; advancing the SI joint implant distally through the cannula lumen and down and into the SI joint with a plunger or tamp; withdrawing the plunger from the cannula, and removing the cannula from the subject while leaving the implant in the patient. An optional second implant may also be implanted adjacent and parallel to the first implant. As set forth above, the procedure herein utilize fewer components and require less time than procedures using other combinations of instruments, such as the system of instruments shown in FIG. 1.

In alternative methods, the cannula may be advanced to the level of the joint (and optionally docked at the joint) and the stylet may then be advanced into the proximal end of the cannula lumen, through the lumen, and advanced into the joint. Or in any of the methods herein, the cannula and stylet may be advanced together to the level of the joint but with the sharp distal end of the stylet not advanced beyond the distal end of the cannula (i.e., still within the cannula lumen), wherein the sharp distal end of the stylet may be advanced distally from the cannula after the combination of cannula and stylet is at or near the level of the joint.

Methods of preparation and implant delivery herein may specifically exclude the use of an obturator, an orientation guide, a decorticator, and one or more guide pins, examples of which are shown in FIG. 1. The methods herein, including claims, may thus be considered to inherently include the negative limitation of not including steps that require or use any of the components herein that are not needed.

A method of positioning any of the SI joint implants herein can include one or more planning steps to determine a desired entry point and/or trajectory. It is understood that figures herein that include planning (including trajectory planning) are exemplary and may not necessarily need to be performed (and not all steps included in any figure are necessarily performed). Additionally, the order of steps may vary to some extent and the implantation process may still be satisfactorily and safely completed.

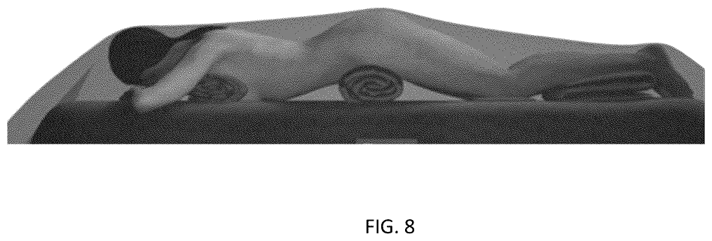

FIG. 8 illustrates an exemplary patient set up and prone positioning for the procedures described herein.

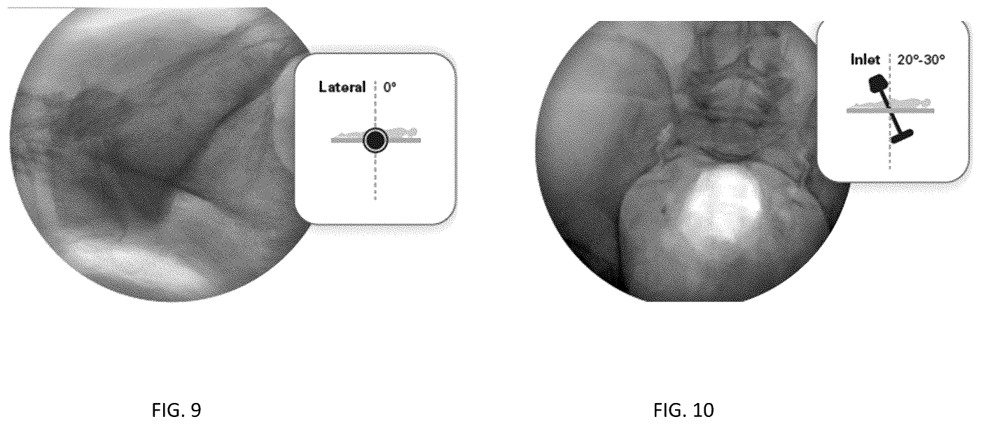

With reference to the exemplary lateral view of FIG. 9, the alar lines can be aligned by superimposing the left and right cortical densities. Next, the sciatic notches are aligned using C-arm swivel. The sciatic notches should remain superimposed throughout the procedure.

With reference to the inlet view of FIG. 10, the fluoroscope is tilted toward the feet until the dense cortical line of the S1-S2 vestigial disc directly overlines the dense cortical line of the sacral promontory. The beam in this view should line up with the anterior cortex of the S1 sacral body.

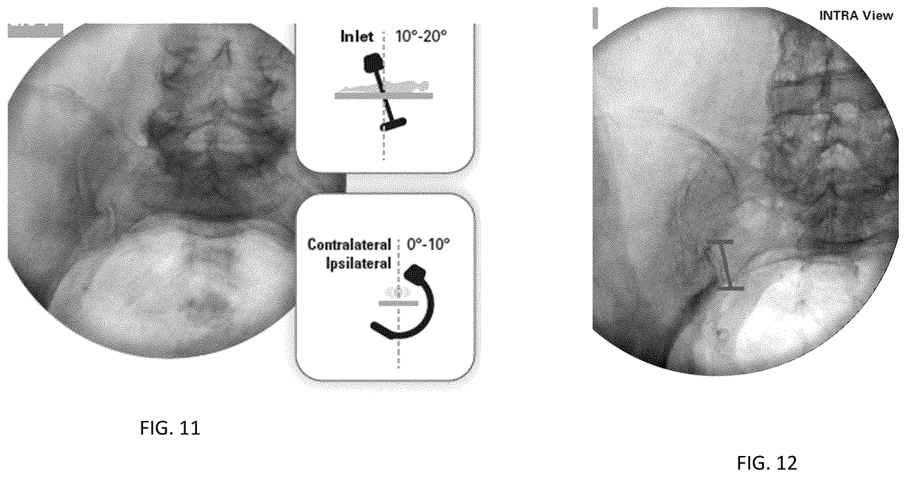

With reference to the INTRA view of FIG. 11 (which is −10 degrees cephalad from the inlet view and slightly oblique ipsilateral or contralateral), the beam in this view will optimize visualization of the posterior aspect of the inferior limb of the articular SI joint. The posterior aspect of the inferior limb of the articular SI joint is the medial joint in this view.

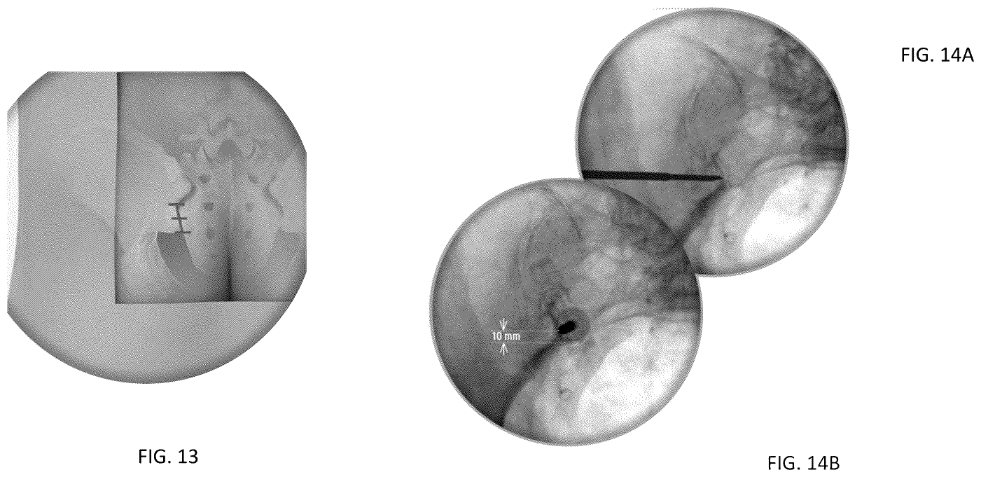

With reference to FIG. 12, in the INTRA view, method can include localizing the inferior aspect of the joint by placing an 8 gauge bone access needle on the skin. Mark on the skin where the medial joint meets the pelvic brim, and trace a 2 cm line posteriorly along the joint, as shown. As shown in FIG. 13, mark the center point, and make an incision at the center marking.

After making an incision, the procedure includes advancing the integrated stylet/cannula (e.g., as shown in FIGS. 4 and 5) to a target position with a target trajectory within the patient, which facilitates placement of the implant through the cannula, as is described below. First, the integrated stylet 412/cannula 410 is placed through the incision and preferably docked in the inferior third of the joint and approximately 10 mm from the anterior pelvic brim, as shown in FIGS. 14A and 14B. The integrated assembly is preferably angled such that it is aligned with the trajectory of the C-arm in the optimized INTRA view, with confirmation that the integrated system is visualized as a dot, as shown in FIG. 14B.

As shown in the trajectory of FIG. 15A (compared with FIG. 15B), the trajectory of the integrated system is adjusted to be parallel to the sciatic notch. The integrated system is advanced approximately 1 cm with tapping of a mallet.

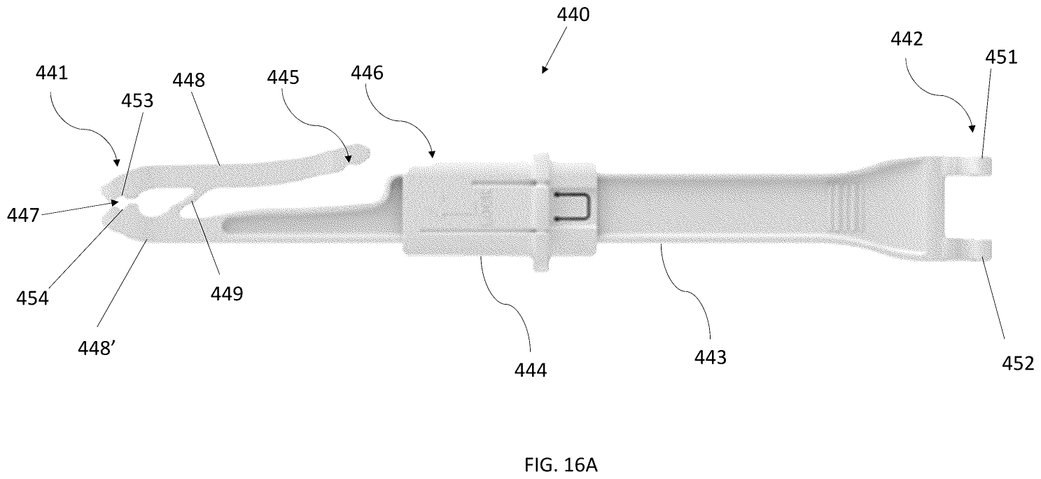

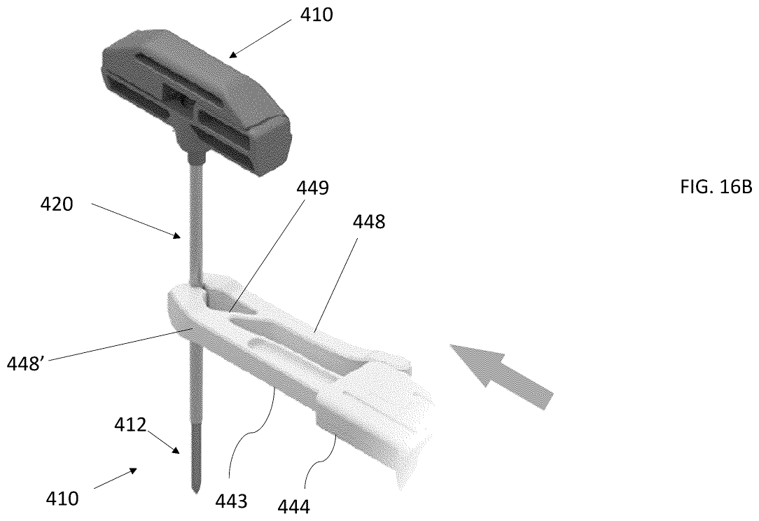

Optionally, tool 440, shown in FIGS. 16A and 16B, may be used to assist with holding and maintaining the positioning and trajectory of the integrated cannula/stylet, as shown in FIG. 16B. Tool 440 is, in this example, a combination function tool with a clamp end 441 and a lever end 442 with first and second hooks (although optionally tool 440 may only be configured as only one of a clamp or lever). The clamp end 441 is configured to clamp and hold the cannula to allow the user to indirectly hold the cannula while keeping their hand away from the radiation source. The movable collar 444 is adapted to be advanced relative to tool elongate body 443 to apply a force to the first end of arm or jaw 448, rotating the arm 448 about pivot 449 and causing the other clamp end of jaw 448 to apply an inward force and securely clamp a cannula within space 447 against the opposing jaw of tool 440. Lock element 445 on arm/jaw 448 is sized and configured to interface with lock element 446 on collar 444 to axially lock the collar in place, maintaining the clamping force around the cannula that is placed in cannula space 447, as shown in FIG. 16B (only a distal portion of tool 440 is shown in FIG. 16B; lever end not shown). The arrow in FIG. 16B represents advancing collar 444 forward to lock the collar in place and maintain a clamping force around cannula 420. Stylet 410, including distal portion 412 extending from cannula 420, is also shown in FIG. 14B. The lever end 442 of tool 440, described in more detail below, includes two hooks that are configured to be positioned in the cavities of the handle end of the stylet (after it's rotated 90 degrees relative to the cannula), and then by pressing down on the body 443 of tool 440, the stylet can be loosened from the bone and removed from the cannula. The combined stylet 410 and cannula 420, as well as tool 440, are described in further detail, with respect to the following exemplary steps of the procedure.

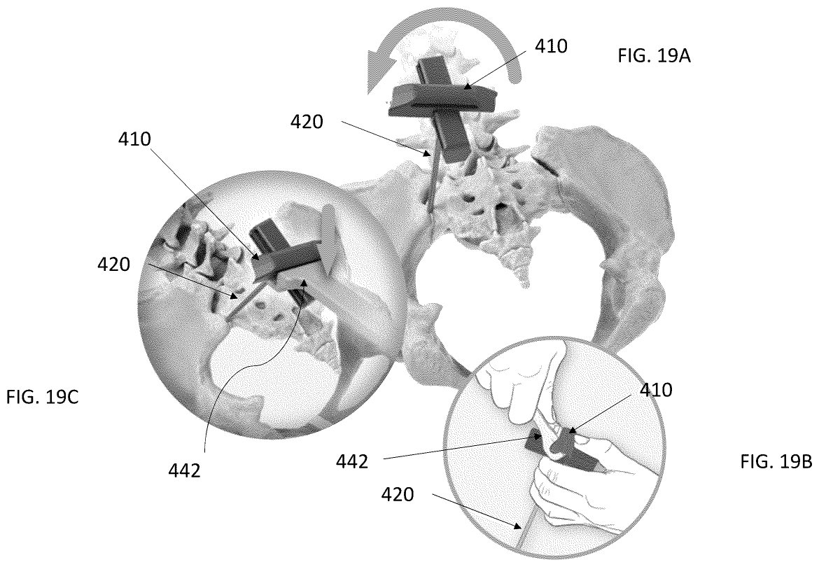

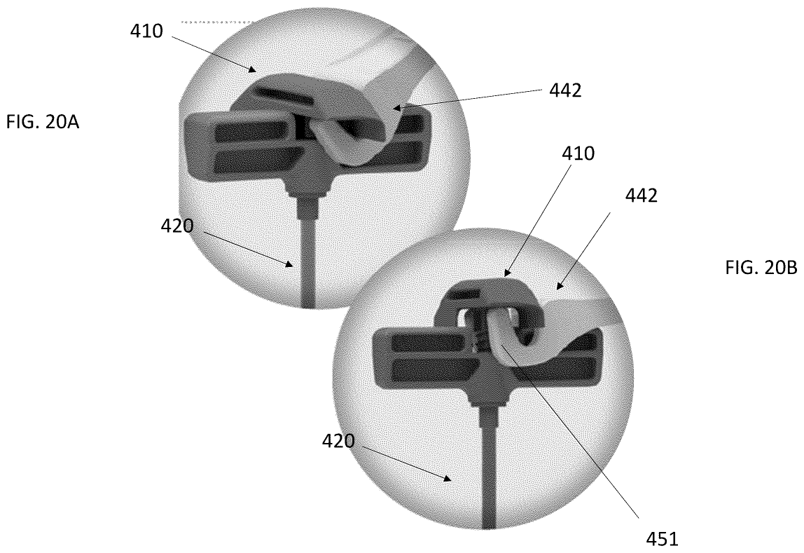

Tool 440 is an example of a tool comprising clamp end 441 comprising a first jaw 448 movable relative to a second jaw 448′ about a pivot 449 (comprising an elongate body as shown), the first jaw 448 including a first locking element 445; a locking collar 444 axially movable relative to the elongate body 443, the locking collar including a second locking element 446 sized and configured to interface with the first locking element 445 to maintain the collar 444 in a locked position relative to the elongate body 443 and prevent clamp ends of the first and second jaws from moving away from one another. Tool 440 also includes a lever end 442 opposite the clamp end 441 along the length of elongate body 443, the lever end 442 comprising first and second hooks 451, 452. Each of hooks 451 and 452 has a curved configuration including a recessed surface as shown, the curved first and second hooks extending from the elongate body, and each of the first and second hooks sized and configured to accommodate and interface with a proximal handle portion of a stylet to facilitate application of a removal force to the proximal handle portion of the stylet, as shown in in FIGS. 19B, 19C, 20A and 20B. As shown in FIG. 16A, each of first and second jaws 448 and 448′ has a recessed surface, 453 and 454 respectively, facing one another (inward) and together defining a cannula space 447, the recessed surfaces 453, 454 each concave (as shown) to interface with an outer surface of a cannula and clamp the cannula in space 447. As shown, pivot 449 comprises an elongate body extending between first jaw 448 and second jaw 448′ at a location proximal to the clamp ends of each of the first and second jaws.

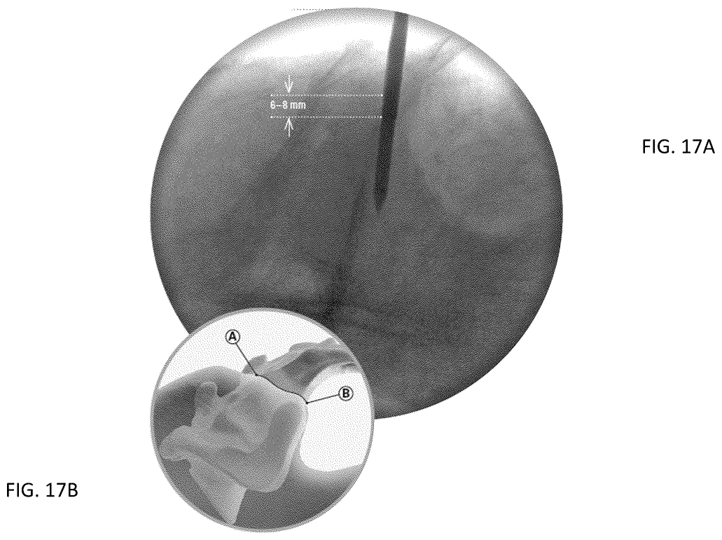

With reference to FIGS. 17A and 17B, the trajectory of the stylet can be confirmed with the lateral view. This may include identifying where the iliac crest intersects the dorsal aspect of the sacrum (location “A” in FIG. 17B); identifying where the sciatic notch intersects the ventral aspect of the sacrum (location “B” in FIG. 17B); the line connecting these two points (as shown in FIG. 17B) is the posterior aspect of the inferior limb of the joint and can be used to determine depth; and advancing the integrated stylet/cannula until the cannula is 6-8 mm below the posterior aspect of the joint. Placing the stylet too deep may result in difficulties removing the stylet from the cannula.

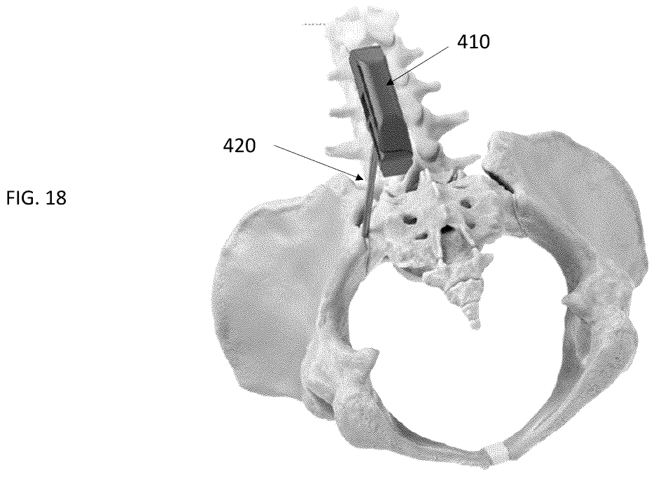

FIG. 18 represents the position and trajectory of the integrated cannula 420/stylet 410 with a distal region of the stylet/cannula within the inferior limb of the SI joint and with the cannula 6-8 mm below the posterior aspect of the joint.

Once the cannula is advanced to the desired depth, and the stylet has prepped the joint, the stylet is removed from the cannula. FIGS. 19A, 19B and 19C illustrate an exemplary stylet loosening and/or removal process, optionally using lever portion 442 of tool 440. The stylet 410 is first turned 90 degrees relative to the cannula 420, as shown by the arrow in FIG. 19A. The hooks of lever portion 442 of tool 440 are then positioned in the cavities of the handle end of the stylet, as shown in FIG. 19B. While holding the cannula with one hand, the other hand applies downward force on tool 440 to loosen the stylet from the bone and remove the stylet from the cannula, as shown in FIG. 19C. FIGS. 20A and 20B provide additional closeup views of the hooks of lever end 442 of tool 440 being inserted into the proximal handle portion of stylet, followed by pressing down on tool 440 to loosen stylet 410 and raise it relative to cannula 420.

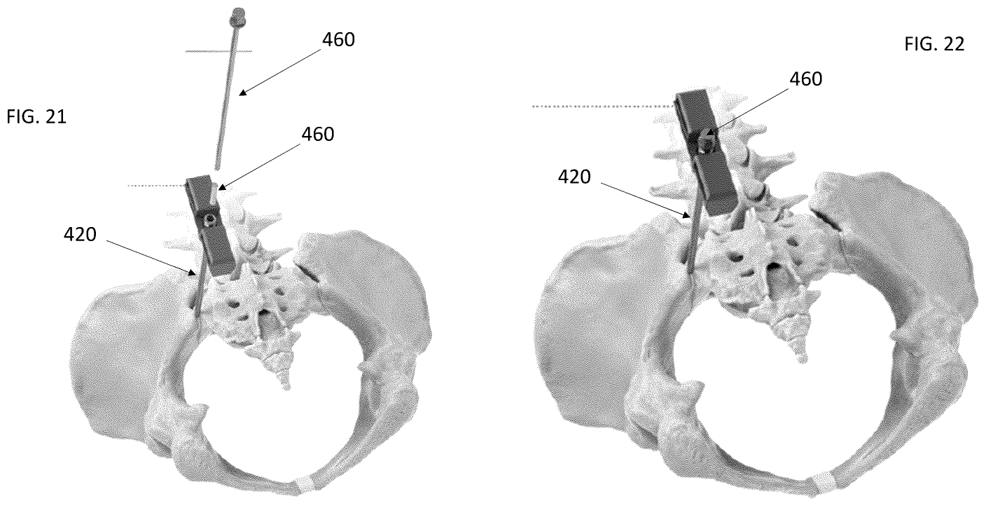

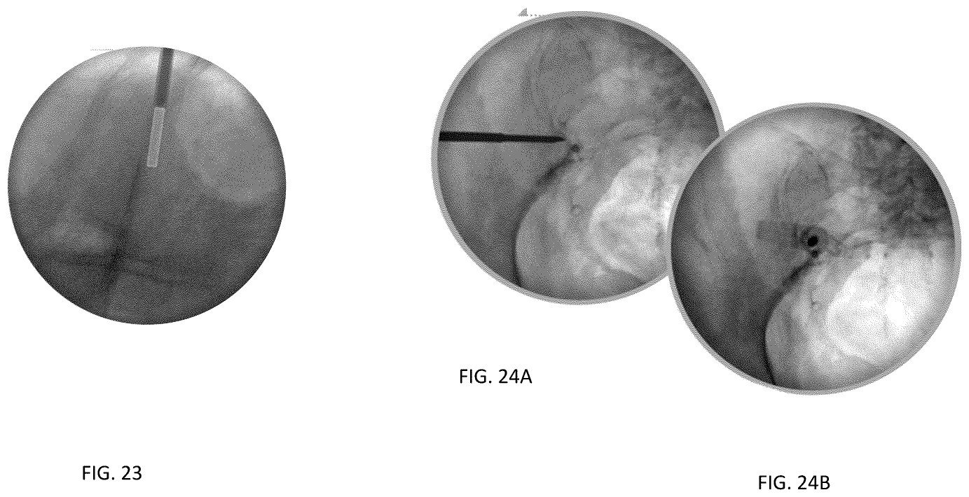

After the stylet has been removed, implant 460 (or other implant herein) is placed into the cannula lumen proximal end, as shown in FIGS. 21 and 22. Tamp or plunger 460 is placed into the cannula lumen and used to push implant 460 through cannula 420 to depth. The position of the cannula 420 should be maintained while advancing the implant to prevent the cannula from being further distally advanced (optionally by holding the cannula with the clamping end of tool 440). The implant may continue to be advanced by gently hitting the proximal end of the tamp 460 with mallet 450 to the positive stop, as shown in FIG. 22. With reference to FIG. 23, the proximal end of the implant should be 6-8 mm below the posterior aspect of the SI joint, which is well short of the iliac cortical densities (ala line).

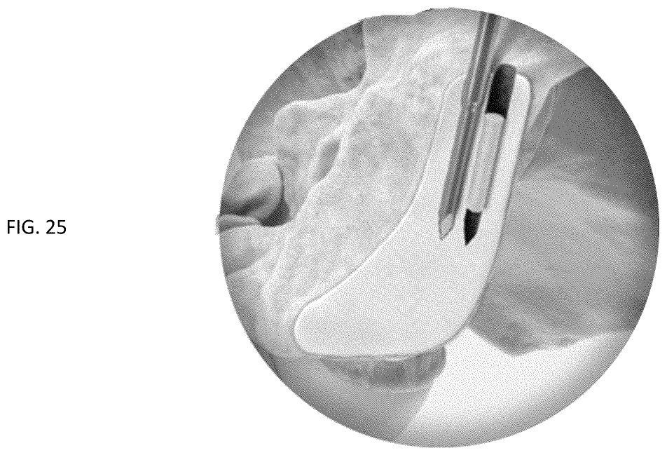

Optionally, any of the procedures herein may include implanting a second implant with a similar procedure to that of the first implant, and parallel to the first implant, which is described with reference to FIGS. 24A, 24B, 25, 26A, 26B, and 26C. With reference to FIGS. 24A and 24B, a second implant should be placed at the junction of the middle third and dorsal third of the SI joint and parallel to the first implant. The stylet is first reinserted into the cannula lumen. In the INTRA view, the integrated stylet/cannula is repositioned to access the junction of the middle third and dorsal third of the SI joint. This is preferably minimum 5 mm of distance center-to-center between the first and second implants. Confirming the cannula/stylet is a dot in the INTRA view when placing the second implant ensures the implants are parallel. The process for implanting the first implant as set forth herein is then repeated for the second implant. FIG. 25 illustrates the stylet/cannula after being advanced adjacent and parallel to the first implant.

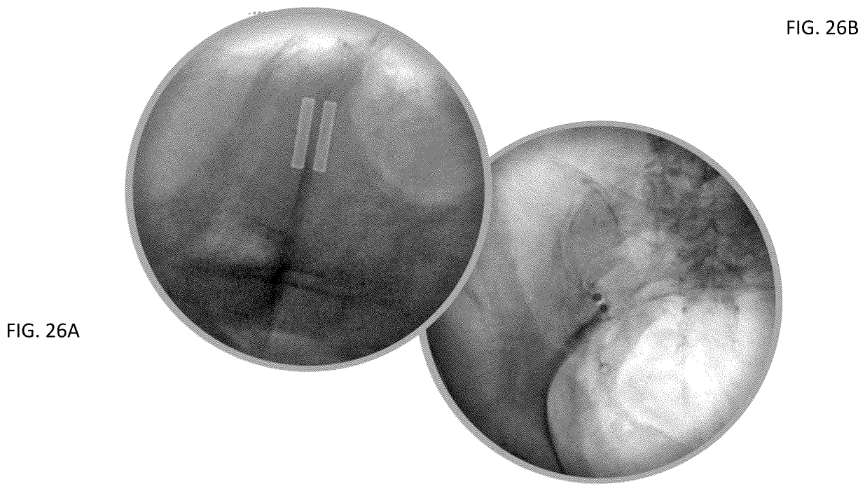

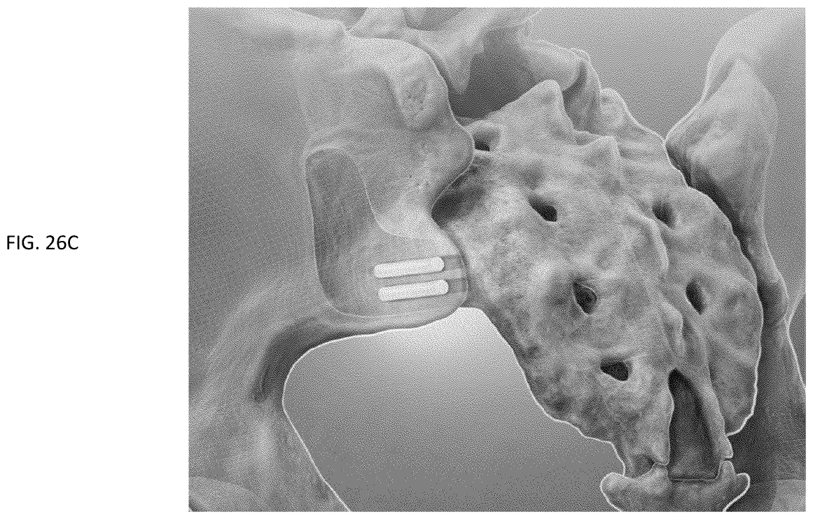

FIGS. 26A and 26B illustrate two views of two implants implanted down the SI joint and parallel (or nearly parallel) to each other as described herein. FIG. 26C is a representation of the final implant location of two implant down the SI joint as described herein, with the preferred trajectory and placement shown in FIG. 15A.

1. A method of positioning a sacroiliac (SI) joint implant, comprising:
in a dorsal trajectory, positioning a stylet removably coupled with a cannula adjacent to an SI joint, the stylet disposed through a cannula lumen with sharp distal tip of the stylet 20 mm-40 mm distally from a cannula distal end;
advancing, in the dorsal trajectory, the sharp distal end of the stylet down and into an SI joint, while a portion of the stylet is within the cannula lumen, to disrupt the SI joint and prepares the SI joint to receive an SI joint implant;
removing the stylet from the SI joint and from the cannula lumen, leaving the cannula distal end adjacent to the SI joint;
positioning an SI joint implant into the cannula lumen;
advancing the SI joint implant with a plunger distally through the cannula lumen and into the SI joint;
removing the plunger from the cannula; and
removing the cannula from the subject while leaving the implant in the SI joint.

2. The method of claim 1, wherein the sharp distal end of the stylet is 25 mm-35 mm distally from the cannula distal end.

3. The method of claim 1, wherein advancing the stylet and cannula comprises advancing the stylet and cannula in a trajectory in line with the ventral cortex of the inferior limb of the articular surface of the SI joint.

4. The method of claim 1, wherein advancing the stylet comprises advancing the stylet until the sharpened distal end of the stylet is caudal and ventral to the iliac cortical density/alar line.

5. The method of claim 1, wherein the method does not include the use of an obturator, a decorticator, and an orientation guide.

6. The method of claim 1, wherein advancing the SI joint implant distally comprises applying an axially directed force on the SI joint implant with the plunger.

7. The method of claim 1, wherein removing the stylet from the SI joint and from the cannula lumen comprises rotating the stylet relative to the cannula, and using a lever end of a tool to loosen the stylet from bone.

8. The method of claim 1, further comprising clamping an outer surface of the cannula with a clamp end of a stabilizing instrument to stabilize the cannula.

9. The method of claim 1, wherein the implant is a first implant, the method further comprising implanting a second implant in the dorsal trajectory, in the SI joint, and parallel to the first implant.

10. The method of claim 9, wherein the first and second implants are implanted in an inferior limb of the SI joint through the articular portion of the SI joint.

11. A method of positioning a sacroiliac (SI) joint implant, comprising
advancing, in a dorsal approach, a cannula to a level of an SI joint;
advancing a sharp distal end of a stylet down the SI joint while a portion of the stylet is within a cannula lumen, wherein a distal tip of the stylet is positioned 20 mm-40 mm distally from a cannula distal end, and wherein advancing the sharp distal end of the stylet down the SI joint disrupts the SI joint and prepares the SI joint to receive an SI joint implant;
removing the stylet from the cannula lumen and leaving the cannula adjacent to the SI joint;
positioning an SI joint implant into the cannula lumen;
advancing the SI joint implant distally through the cannula lumen and down and into the SI joint; and
removing the cannula from the subject while leaving the implant in the patient.

12. A system for implanting a sacroiliac (SI) joint implant down the SI joint in a dorsal approach, comprising:
a cannula having a lumen and a sharpened stylet removably couplable to the cannula, the cannula and stylet are each sized and configured such that, when coupled, a portion of the stylet is within the cannula lumen and a distal tip is 20 mm to 40 mm distal to a distal end of the cannula.

13. The system of claim 12, wherein the distal tip is 25 mm to 35 mm distal to a distal end of the cannula.

14. The system of claim 12, wherein the cannula lumen includes one or more inner threads formed therein and the stylet includes one or more outer threads adapted to mate with the cannula lumen inner threads.

15. The system of claim 12, further comprising an implant plunger sized and configured, relative to the cannula lumen, to be advanced through the cannula lumen to advance an SI joint implant disposed in the cannula lumen.

16. The system of claim 12, wherein the system excludes an obturator, a decorticator, and an orientation guide, and wherein the system is packaged without the obturator, the decorticator, and the orientation guide.

17. A cannula stabilization and stylet removal tool, comprising:
an elongate body adapted to be held by a hand of an operator;
a clamp end comprising a first jaw movable relative to a second jaw about a pivot, the first jaw including a first locking element;
a locking collar axially movable relative to the elongate body, the locking collar including a second locking element sized and configured to interface with the first locking element to maintain the collar in a locked position relative to the elongate body and prevent clamp ends of the first and second jaws from moving away from one another; and
a lever end opposite the clamp end, the lever end comprising first and second hooks each having a curved configuration including a recessed surface, the curved first and second hooks extending from the elongate body, and each of the first and second hooks sized and configured to accommodate and interface with a proximal handle portion of a stylet to facilitate application of a removal force to the proximal handle portion of the stylet.

18. The tool of claim 17, wherein the first and second jaws each have a recessed surface facing one another and together defining a cannula space, the recessed surfaces each concave to interface with an outer surface of a cannula.

19. The tool of claim 17, wherein when the collar is locked, and in the absence of a cannula in a cannula space between clamp end of each of the first and second jaws, a distance between the first and second jaws is maintained.

20. The tool of claim 17, wherein the pivot comprises an elongate body extending between the first and second jaws proximal to the clamp ends of each of the first and second jaws.
